(19) United States
(12) Patent Application Publication
(10) Pub. No.: US 2015/0102123 A1
(43) Pub. Date: Apr. 16, 2015

(54) Retained cap aromatherapy container

(71) Applicant: Roy L. Nichols, Enterprise, AL (US)

(72) Inventors: Roy L. Nichols, Enterprise, AL (US); Sin-Hsiung Chen, Tainan City (TW); Chia-Te Chen, Tainan City (TW)

(73) Assignee: Roy L. Nichols, Enterprise, AL (US)

(21) Appl. No.: 14/102,583

(22) Filed: Dec. 11, 2013

Related U.S. Application Data
(60) Provisional application No. 61/889,135, filed on Oct. 10, 2013.

(51) Int. Cl. A61L 9/12

(52) U.S. Cl. CPC A61L 9/127
USPC 239/44; 239/34

(57) ABSTRACT

A retained cap aromatherapy container has a cap, a base and an essential oil reservoir. The base has an inner sleeve with an opening in it that fits into the cap. When the container is closed, the opening is covered and aromatherapy vapors are contained. When the container is opened, the cap moves up the sleeve and the opening is revealed. This allows vapors to escape, but the cap is retained on the sleeve. A peg on the sleeve may engage an angled groove in the cap so that the container is opened by twisting the cap. Outer caps and outer bases may snap over inner caps and inner bases to provide a vapor seal as well as provide an aesthetically pleasing surface.

### CROSS-REFERENCE TO RELATED APPLICATION

This application claims priority to copending US provisional patent application “Retained cap aromatherapy container”, Ser. No. 61889135, filed Oct. 10, 2013. Said application is incorporated herein by reference in its entirety.

### COPYRIGHT NOTICE

A portion of the disclosure of this patent document contains material to which a claim for copyright is made. The copyright owner has no objection to the facsimile reproduction by anyone of the patent document or the patent disclosure, as it appears in the Patent and Trademark Office patent file or records, but reserves all other copyright rights whatsoever.

### BACKGROUND OF THE INVENTION

An essential oil is “a concentrated hydrophobic liquid containing volatile aroma compounds from plants.”(Wikipedia “Essential Oils”, viewed Oct. 1, 2013). Examples of essential oils include lavender oil, peppermint oil, ginger oil, spearmint oil and mixtures thereof. They have been used in aromatherapy where healing effects have been attributed to inhaling the volatile compounds. Different inhalers have been designed to allow a person to travel around with essential oils in a closed chamber and then open the chamber to inhale the aromas as needed. FIGS. 1A, 1B and 1C illustrate different views of a prior art inhaler. The images are taken from U.S. D448,153 S, “Container for Aroma Therapy Oils”.

FIG. 1A is a front view of closed container 100. The container comprises a cap 102 and a base 104. The oils are housed in the base. FIG. 1B is a front view 110 of the container with the cap 112 removed from the base 116. An inhaler 114 is exposed and volatile aromas 118 can be inhaled. FIG. 1C is a top view of the base 120 of FIG. 1B showing holes 122 in the inhaler which allow for vapors to diffuse from the oils therein.

One of the drawbacks of the prior art design is that the cap is separated from the base when the container is opened and hence can be lost. Another drawback is that the seal between the cap and base may not be air tight and thus vapors will escape and be smelled even if the container is closed. A further limitation is that only one oil mixture may be carried in the container. There is need, therefore, for an essential oil container that:
has a cap that can be opened without being separated from the base;
can provide an air tight seal when closed; and
has the capability of providing more than one essential oil mixture in the same container.

### SUMMARY OF THE INVENTION

The summary of the invention is provided as a guide to understanding the invention. It does not necessarily describe the most generic embodiment provided herein.

FIG. 2A is a front view of an exemplary embodiment of a retained cap aromatherapy container 200. The container comprises a cap 202 and a base 204. An essential oil reservoir 206 is located in the cap. It may be alternatively located in the base. The reservoir may comprise a porous structure, such as a cotton wad or sintered plastic powder, which would retain a dose of essential oil. The base may comprise a chamber 208. The chamber may house air with essential oil vapors diffused therein from the reservoir. Thus when the container is opened, there is a volume of air with oil vapors readily available for inhaling.

The overall length 214 of the container may be a typical hand width, such as about 10 cm. The length of the cap 216 is suitable for a typical person to grab with his or her fingers, such as about 3.3 cm. The width 212 is suitable for a person to hold in his or her hand, such as about 1.8 cm. One or more indentations 203, bumps 205, or other forms of surface roughness, such as knurling, may be provided to help a person grasp the cap with his or her fingers when opening it or closing it.

FIG. 2B is a front view of container 220 of FIG. 2A with the cap opened. Pulling the cap open reveals an inner sleeve 222. The sleeve has one or more openings 224 which allow essential oil vapors 226 to be inhaled by a user. The openings may be holes, screens, porous material or solid materials that are permeable to the essential oil vapors, such as semipermeable membranes. The sleeve comprises a stop 228 which prevents the total removal of the cap. The stop may be clickable so that when the cap is pulled up to the stop it is held in position. Thus when the container is pulled open and clicked, it may be held in one hand and the essential oil vapors inhaled. The container may also comprise a seal 229 so that the cap forms an airtight closure when closed.

The seal may also be clickable so that the cap is held firmly in place when closed. The strength of the clickable stop and clickable seal should be high enough to hold the cap in place when opened or closed, but low enough so that a person of compromised strength, such as a person recovering from an operation or a person with arthritis, can open and close it.

The materials of construction of the container should be physically and aromatically compatible with the essential oils housed therein. Polypropylene (PP) and polyethylene (PE) are suitable for parts of the container that come in contact with the essential oils. Neither PP nor PE will impact the aroma of the oils and are stable against cracking when exposed to the oils. Acrylonitrile Butadiene Styrene (ABS), Acrylonitrile Styrene (AS) and Acrylic materials are suitable for parts of the container that do not come in direct contact with the essential oils. These materials have a glossy finish after molding. They are also suitable for vacuum plating of metals to give a metallic finish. Metals, such as aluminum and stainless steel, ceramics and glass are also suitable materials of construction for the container. The above mentioned materials may be suitable for the seal. Elastomeric materials, such as silicone rubber, may also be used for the seal.

### BRIEF DESCRIPTION OF DRAWINGS

FIG. 1A is a front view of a closed prior art container.

FIG. 1B is a front view of an open prior art container.

FIG. 1C is a top view of the base of a prior art container.

FIG. 2A is a front view of a closed retained cap aromatherapy container.

FIG. 2B is a front view of an open retained cap aromatherapy container.

FIG. 3 is a perspective exploded view of a twist-open aromatherapy container.

FIG. 4A is a cross section exploded view of the top of the twist-open aromatherapy container of FIG. 3.

FIG. 4B are top or bottom views of components of the same twist-open aromatherapy container.

FIG. 5A is a front view of an inner cap of the same twist-open aromatherapy container.

FIG. 5B is a 90 degree rotated front view of the inner cap of FIG. 5A.

FIG. 6 is a cross section exploded view of the top of a double twist-open aromatherapy container.

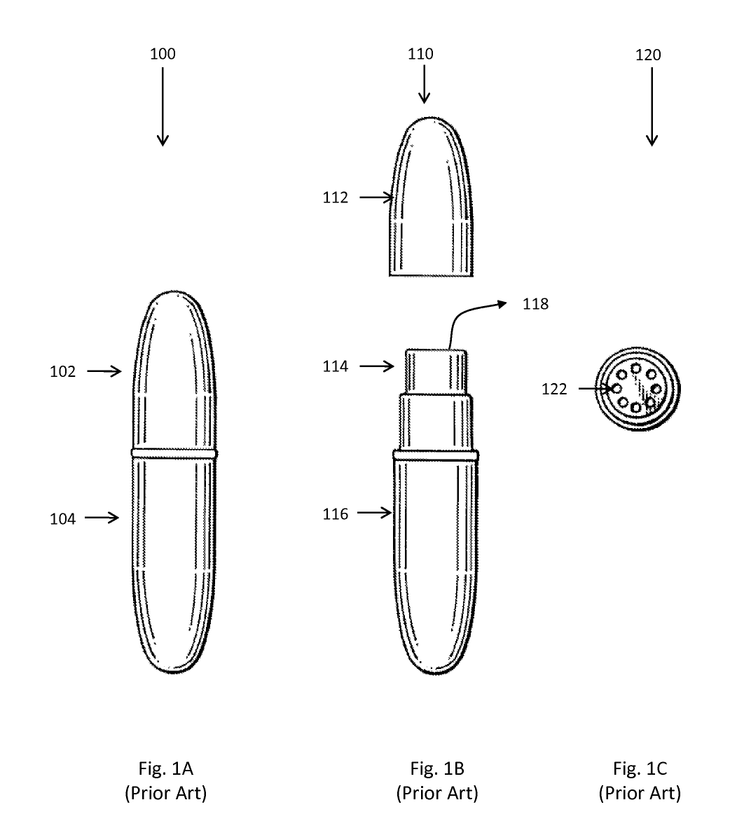

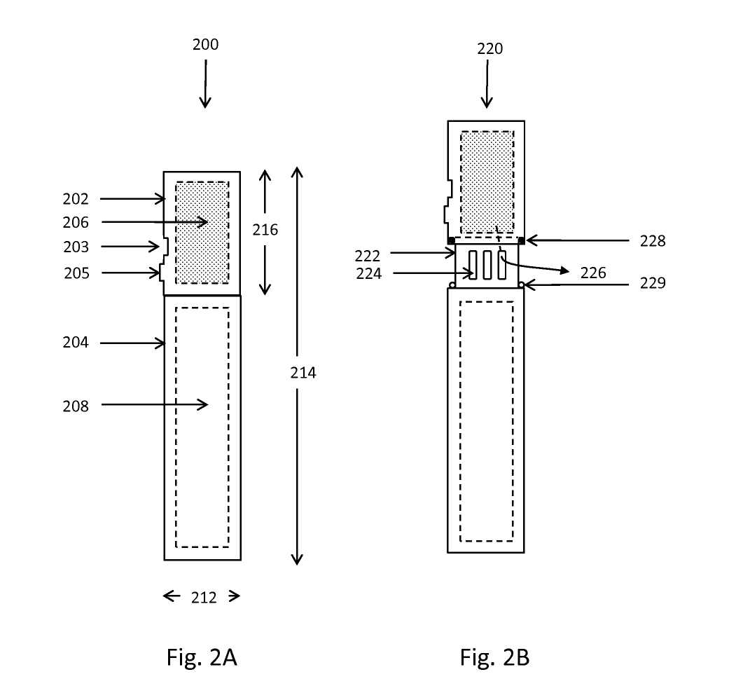

### DETAILED DESCRIPTION

The detailed description describes non-limiting exemplary embodiments. Any of the individual features disclosed may be combined with other disclosed features as required by different applications for at least the benefits described herein. As used herein, the term “about” means plus or minus 10% of a given value unless specifically indicated otherwise.

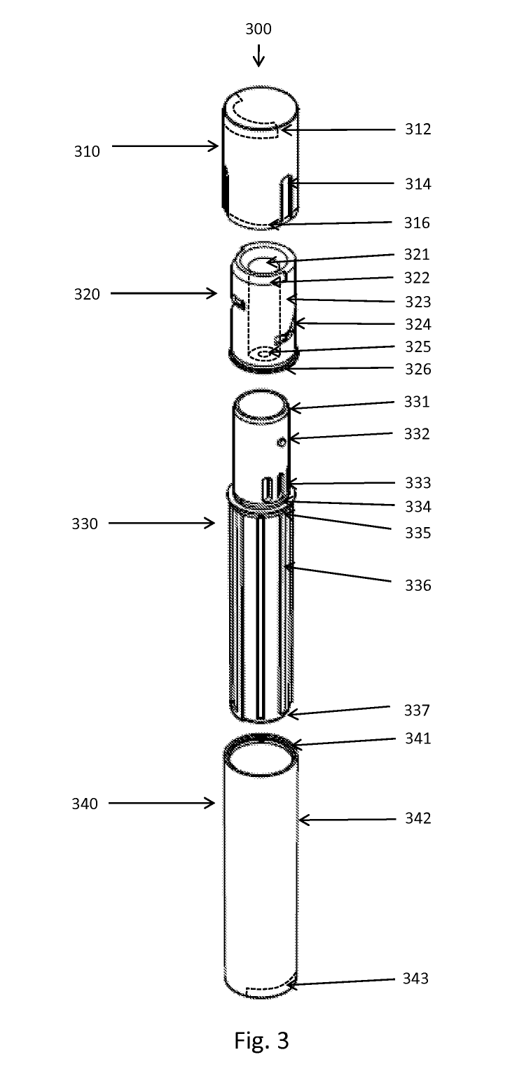

FIG. 3 is a perspective exploded view of an embodiment of a twist-open aromatherapy container 300. The twist-open aromatherapy container comprises an outer cap 310, inner cap 320, inner base 330 and outer base 340.

The outer cap comprises an outer cap chamfer 312, a grip 314 and an outer cap snap 316. The outer cap chamfer and outer cap snap are internal to the outer cap.

The inner cap comprises a wick chamber 321, an inner cap chamfer 322, a sleeve channel 323, one or more peg grooves 324, and an inner cap snap 326. The bottom of the wick chamber of the inner cap comprises a hole 325 to permit essential oil vapors to pass therethrough.

The inner base comprises a sleeve 331, one or more pegs 332, one or more vents 333, a vapor seal 334, an inner base snap 335, one or more ribs 336 and an inner base chamfer 337.

The outer base comprises an outer base snap 341, a wall 342 and an outer base chamfer 343. The inner base chamfer is internal to the inner base.

As used herein, a “chamfer” is an alignment surface. For example, when the inner cap chamfer is urged towards the outer cap chamfer, the angled surfaces will cause the inner cap and outer cap to align. This will assist in the proper orientation of the outer cap relative to the inner cap for subsequent processing, such as printing.

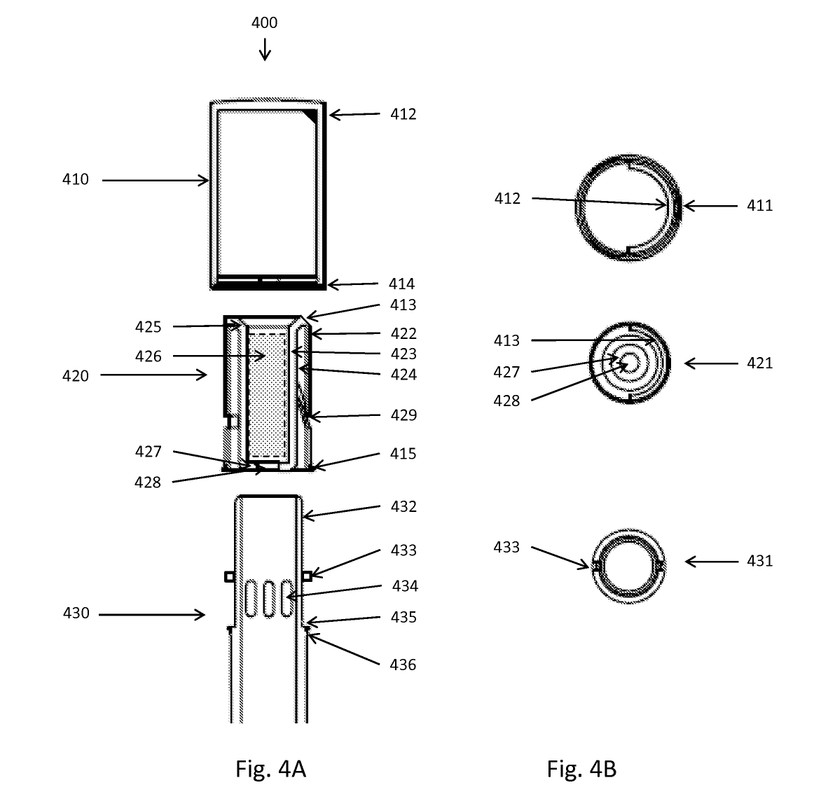

As used herein, a “snap” is a joining surface. The inner cap snap, for example, may comprise a protruding ring (item 415, FIG. 4A). The outer cap snap may comprise a recessed groove (item 414, FIG. 4A). Thus when the inner cap snap is urged towards the outer cap snap, the protruding ring of the inner cap snap nests within the recessed groove of the outer cap snap and the inner cap and outer cap are thereby joined.

When the twist-open container is in its assembled state:
a wick (item 426, FIG. 4A) with essential oil or other aromatic material is located within the wick chamber;
the outer cap is joined to the inner cap such that the outer cap chamfer and inner cap chamfer are aligned and the outer cap snap is engaged with the inner cap snap. Thus when the outer cap is twisted, the inner cap will twist as well under the influence of the mated chamfers and engaged snaps;
the inner cap is mounted on the inner base such that the sleeve of the inner base resides within the sleeve channel of the inner cap and the one or more pegs of the inner base engage the one or more peg grooves of the inner cap; and
the outer base is joined to the inner base such that the base chamfers are aligned and the base snaps are engaged.

Thus when the outer cap is twisted relative to the outer base, the inner cap will move up or down relative to the outer base thus blocking or revealing the vents. If the cap is twisted open, the vents are revealed, the vapors from the essential oils in the wick pass through the hole in the bottom of the wick chamber and out of the vents to a user. If the cap is twisted closed, the vents are blocked, the vapor seal is engaged and the essential oil vapors are sealed within the aromatherapy container.

FIGS. 4A and 4B are magnified exploded views of the top of the twist-open aromatherapy container of FIG. 3. The views of FIG. 4A are longitudinal cross sections. The views of FIG. 4B are top or bottom views. The outer cap 410, 411 inner cap 420, 421 and top portion of the inner base 430, 431 are shown. The outer cap chamfer 412 is shown as an angled cross section in FIG. 4A and as a semicircular element in FIG. 4B. The corresponding inner cap chamfer 413 is shown as a beveled edge in FIG. 4A and as a semicircular element in FIG. 4B. When the two chamfers are mated, then the outer cap and inner cap are rotationally aligned. Alternatively sized and shaped chamfers are suitable provided they engage with each other such that the outer and inner caps are rotationally aligned when urged together. For example, the chamfers can span more or less than the 180 degrees as illustrated in FIG. 4B. A span in the range of 30 degrees to 330 degrees is suitable.

Cross sections of the outer cap snap 414 and inner cap snap 415 are shown in FIG. 4A. The outer cap snap comprises a recessed channel. The inner cap snap comprises a protruding ring. Force is required to join the snaps. Force is also required to separate the snaps once they are engaged. Any geometry requiring force to engage and force to separate may be suitable.

The inner cap comprises an outer wall 422, and a wick chamber 423. The wick chamber has a flared top opening 425. This facilitates the insertion of an essential oil wick 426. The wick chamber further comprises a bottom ledge 427. This holds the essential oil wick in the wick chamber. The bottom ledge describes an opening 428 which allows the passage of essential oil vapors to the area below the wick chamber.

The wick may be any geometry that holds essential oils. This includes a porous wick of sintered powder, a wad of fiber, such as a cotton ball, or an adsorbent solid, such as a molecular sieve or activated charcoal, or an absorbent solid, such as a sponge.

The outer wall and wick chamber describe a sleeve channel 424. The sleeve channel receives the sleeve 432 of the inner base. The outer wall further describes a peg groove 429. The peg groove receives a peg 433 located on the sleeve of the inner base.

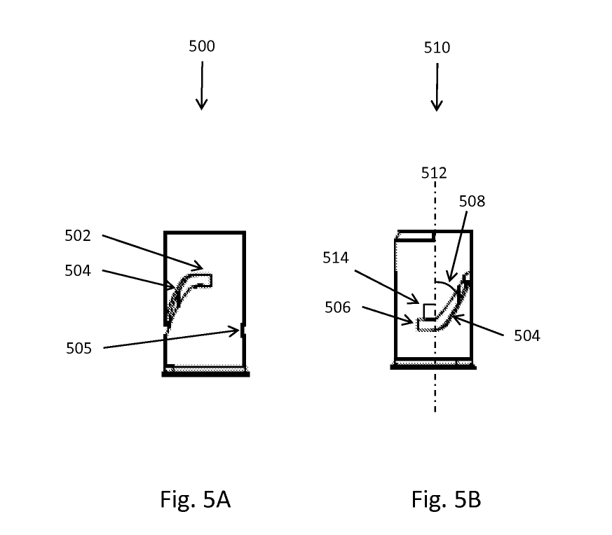

Referring to FIGS. 5A and 5B, a central portion of the peg groove 504 is at an angle 508 with respect to the longitudinal axis 512 of said the inner cap such that the inner cap will move up or down relative to the base when the inner cap is twisted about said longitudinal axis. The ends of the peg groove 502 and 506 may be at a normal or acute angle 514 with respect to the longitudinal axis of said inner cap such that the inner cap can be forcibly retained in either an open configuration or closed configuration.

Referring back to FIGS. 4A and 4B, the sleeve of the inner base describes at least one vent 434. The inner cap and inner base are dimensioned such that the vent is alternatively revealed and blocked when the inner cap is rotated and moves up and down. Different combinations of vent configuration and peg groove configurations are suitable. FIG. 4A illustrates three vents on one side of the inner base. Three additional vents may be provided on the other side of the base. There may be two or more peg grooves and pegs. The peg grooves may be dimensioned such that their central portions span about ¼ of the circumference of the outer wall of the inner cap. Thus the vents will be revealed or closed by about ¼ turn of said inner cap. This makes the aromatherapy container easy to open and close.

A vapor seal 435 may be provided on the inner base. The vapor seal may be a flat ledge that the inner cap presses against when the aromatherapy container is closed. It may also comprise an elastomer, such as silicone rubber.

An inner base snap 436 may be provided to join the inner base to the outer base (item 340 FIG. 3).

FIG. 5A shows a front view of an inner cap 500. FIG. 5B shows a front view 510 of the inner cap rotated by 90 degrees. The peg groove comprises a top flat end 502, a middle angled portion 504 and a bottom flat end 506. The top flat end allows the inner cap to exert a stable downward force when closed. The bottom flat end allows the inner cap to exert a stable upward force when opened. Thus when closed, the inner cap can exert a stable force against the vapor seal. When opened, the inner cap will remain open with a stable force.

The inner cap illustrated in FIGS. 5A and 5B has a second peg groove 505 opposite the first groove and dimensioned similarly. There is also a corresponding second peg on the opposite side of the inner base. Thus the forces exerted by the pegs on the inner cap will be horizontally balanced.

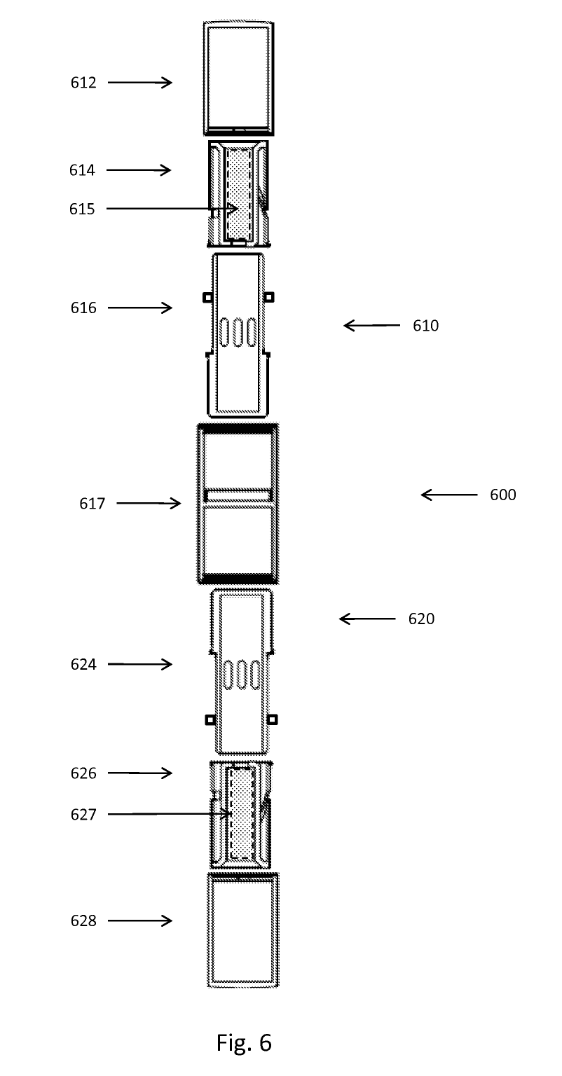

FIG. 6 is an exploded view 600 of a double twist-open aromatherapy container. Cross sections are shown. The double container comprises a first outer cap 612, first inner cap 614, first wick 615 and first inner base 616, a partitioned outer base 617, a second inner base 624, a second inner cap 626 a second wick 627 and a second outer cap 628. Thus when assembled, there is a first aromatherapy container 610 joined back-to-back with a second aromatherapy container 620. Different essential oils can be placed in the different wicks and a user can select which aromatherapy oils to partake of. The inner bases are shortened relative to the inner base of the container of FIG. 3 so that the overall length of the double aromatherapy container is about the same as the overall length of the single aromatherapy container.

Wherein we claim:

1. An aromatherapy container, said container comprising:
a) a first cap;
b) a first base; and
c) a first essential oil wick wherein:
d) said base comprises an inner sleeve;
e) said sleeve comprises an openings;
f)
g) said first cap sits over said sleeve when said container is closed such that said opening is blocked by said first cap;
h) said first cap is retained on said sleeve when said container is opened such that:
i) said opening is unblocked; and
ii)
iii) said first cap remains attached to said base; and,
i) said first cap comprises an inner cap comprising:
i) an inner cap outer wall; and
ii) a wick chamber;
wherein:
iii) said inner cap outer wall is joined to said wick chamber to form a sleeve channel; and
iv) said sleeve resides within said sleeve channel;
v) said wick chamber comprises a horizontally extended bottom ledge;
vi) at least a portion of said wick is within said wick chamber; and
vii) said bottom ledge is adapted to hold said wick in said wick chamber when said opening is unblocked.

2. (canceled)

3. The aromatherapy container of claim 1 wherein:
a. said sleeve comprises a first peg;
b. said inner cap outer wall comprises a first peg groove;
c. said peg is engaged within said peg groove;
d. said peg groove comprises a central portion which is at an angle with respect to the longitudinal axis of said inner cap such that said inner cap will move up or down relative to said base when said inner cap is twisted about said longitudinal axis.

4. The aromatherapy container of claim 3 wherein:
a. said peg groove comprises an end portion wherein said end portion is at a normal or acute angle with respect to said longitudinal axis; and
b. said central portion of said peg groove radially spans about ¼ of the circumference of said inner cap outer wall.

5. The aromatherapy container of claim 3 which comprises a second peg opposite said first peg on said sleeve and a second peg groove opposite said first peg groove on said inner cap.

6. (canceled)

7. The aromatherapy container of claim 16 wherein:
a. said outer cap comprises an outer cap snap;
b. said inner cap comprises an inner cap snap; and
c. said outer cap snap is engaged with said inner cap snap.

8. The aromatherapy container of claim 16 wherein:
a. said outer cap comprises an outer cap chamfer;
b. said inner cap comprises an inner cap chamfer; and
c. said outer cap chamfer is mated to said inner cap chamfer such that said inner cap moves with said outer cap when said outer cap is twisted relative to said longitudinal axis.

9. The aromatherapy container of claim 16 wherein:
a. said base comprises an outer base and an inner base;
b. the length of said container is about 10 cm;
c. the width of said container is about 1.8 cm;
d. the length of said first cap is about 3.3 cm; and
e. said outer cap comprises a grip to assist a person in grasping it.

10. The aromatherapy container of claim 9 wherein said grip is an indentation.

11. The aromatherapy container of claim 1 wherein said wick comprises wad of fiber.

12. The aromatherapy container of claim 11 wherein said wad of fiber is a cotton ball.

13. The aromatherapy container of claim 1 wherein:
a. said wick comprises an essential oil;
b. said essential oil comprises one or more of lavender oil, peppermint oil, ginger oil, spearmint oil and mixtures thereof; and
c. said wick chamber comprising one or more of polypropylene or polyethylene.

14. The aromatherapy container of claim 1 which comprises:
a. a second cap;
b. a second base; and
c. a second essential oil reservoir
wherein said first base and said second base are joined together by a partitioned outer base such that:
d. the length of said container is about 10 cm; and
e. the width of said container is about 1.8 cm.

15. The aromatherapy container of claim 14 wherein:
a. the length of said first cap is about 3.3 cm; and
b. the length of said second cap is about 3.3 cm.

16. The aromatherapy container of claim 1 which further comprises an outer cap and wherein:
a. said wick chamber has an open top;
b. said open top is dimensioned to receive said wick; and
c. said outer cap covers said open top.
